(19) United States
(12) Patent Application Publication
(10) Pub. No.: US 2017/0340252 A1
(43) Pub. Date: Nov. 30, 2017

(54) DEVICE FOR OPTICAL MEASUREMENT OF LIVING BODY, ANALYSIS DEVICE, AND ANALYSIS METHOD

(71) Applicant: HITACHI, LTD., Tokyo (JP)

(72) Inventors: Masahi KIGUCHI, Tokyo (JP); Tsukasa FUNANE, Tokyo (JP)

(73) Assignee: HITACHI, LTD., Tokyo (JP)

(21) Appl. No.: 15/537,686

(22) PCT Filed: Dec. 22, 2014
(86) PCT No.: PCT/JP2014/083925

§ 371 Date: Jun. 19, 2017

(51) Int. Cl. A61B 5/1455; A61B 5/00

(52) U.S. Cl. CPC A61B 5/14552; A61B 5/14553; A61B 5/6814

(57) ABSTRACT

A device is provided for the optical measurement of a living body. For the purpose of separating a signal coming from the change in hemodynamics in a deep part from a signal coming from the change in hemodynamics in skin, light irradiation sites and light detection sites are positioned so that the measurement is achieved employing at least two SD distances. At two SD distances, the change in a logarithmic value of the detected light at every time point is determined employing a logarithmic value of the amount of detected light under specific conditions or at a specific time point as a starting point. A gradient value for a differential SD distance, which is a difference between the amount of change obtained by the measurement at a longer SD distance and the change obtained by measurement at a shorter SD distance, is used as a measurement amount.

### TECHNICAL FIELD

The present invention relates to a technique for accurately measuring and analyzing living body internal information such as a hemodynamic change in brain, in a device for the optical measurement of a living body.

### BACKGROUND ART

A brain function measurement device using near infra-red spectroscopy (NIRS) can be used for medical and research equipment, confirmation of the effect of education and rehabilitation, health management at home, or market research such as product monitoring. In addition, the brain function measurement device can be used for measurement of oxygen saturation in tissue and measurement of oxygen metabolism in muscle by the same method. Further, the brain function measurement device can be used for a general absorption spectroscopic apparatus for measurement of a light scatterer as a measurement target, the measurement including measurement of sugar content in a fruit.

In brain function measurement using NIRS of the related art, in order to observe a local hemodynamic change near a surface layer of a human brain in a non-invasive manner, a subject is irradiated with light having a wavelength in a range from the visible region to the infrared region, an amount of light which is passed through the inside of the subject is measured at a position separated at a distance of several centimeters from a light irradiation position, an amount of change in the product of a hemoglobin concentration and an optical path length (hereinafter, referred to as ΔCL) is measured using the modified Lambert-Beer equation. That is, in the NIRS measurement, the change in the amount of detected light which is passed through the living body is a direct measurement amount, and ΔCL is an indirect measurement amount. In a clinical field, measurement for language functions, visual functions, or the like is performed using this method. As techniques related to these method in the related art, there are the following PTL 1 to PTL 3.

### CITATION LIST

### Patent Literature

PTL 1: JP-T-2005-533609

PTL 2: JP-A-59-207131

PTL 3: WO2012/005303

### SUMMARY OF INVENTION

### Technical Problem

In the NIRS measurement, since an optical path length L depends on a distance between the light irradiation position and the light detection position (hereinafter, referred to as an SD distance), ΔCL also depends on the SD distance. For this reason, there is a problem that a measurement amount differs between devices with different SD distances. Conversely, in order to compare measurement data, it is necessary to dispose the light irradiation position and the light detection position such that the SD distances become the same. Thus, there is a problem that the measurement positions for a brain are displaced between subjects with different head shapes and different head sizes.

Furthermore, since a brain is irradiated with light from above scalp, there is a possibility that the measurement data is influenced by a hemodynamic change in skin of scalp, and thus a method of extracting and removing such skin blood flow components is studied. For example, in PTL 1 and PTL 2, measurement at SD distances is performed, and a signal coming from the change in hemodynamics in skin is removed by subtracting, from the measurement signal at the long SD distance, a value obtained by multiplying the measurement signal at the short SD distance by an appropriate coefficient. In addition, in PTL 3, a signal coming from the change in hemodynamics in a deep part is separated and obtained from a signal coming from the change in hemodynamics in skin, by using the fact that the amplitude of a hemodynamic signal in skin and the amplitude of a hemodynamic signal in a deep part differ in SD distance dependence. In any method, since the indirect measurement amount is ΔCL, the problem that the signal amplitude depends on the SD distance is not solved.

An object of the present invention is to provide a device for the optical measurement of a living body, an analysis device, and an analysis method capable of obtaining a value proportional to a hemodynamic change at a deep part, that is, a value corresponding to a concentration change of an absorber inside a living body, regardless of the SD distance.

### Solution to Problem

In order to achieve the above-mentioned object, the present invention provides a device for the optical measurement of a living body, including: one or more light irradiators that irradiate a light irradiation position on the living body with light; one or more light detectors that detect, at a light detection position on the living body, light which is propagated through the living body; and an analysis unit that analyzes a detection signal obtained by the one or more light detectors, in which the analysis unit obtains, based on the detection signal, a value corresponding to a concentration change of an absorber inside the living body by using a gradient value with respect to a distance between the light irradiation position and the light detection position, the gradient value being a differential value in an amount of change between a logarithmic value of received light intensity measured by a set of the light irradiation position and the light detection position which are disposed at a distance d1 and a logarithmic value of received light intensity measured by a set of the light irradiation position and the light detection position which are disposed at a distance d2 or being a differential value in an amount of change of hemoglobin, the sets of the light irradiation position and the light detection position being disposed on a surface of tissue of the living body.

In addition, in order to achieve the above-mentioned object, the present invention provides an analysis device including: an analysis unit that analyzes a detection signal obtained by detecting, at a light detection position on a living body, light which is irradiated from a light irradiation position on the living body and is propagated through the living body, in which the analysis unit obtains, based on the detection signal, a value corresponding to a concentration change of an absorber inside the living body by using a gradient value with respect to a distance between the light irradiation position and the light detection position, the gradient value being a differential value in an amount of change between a logarithmic value of received light intensity measured by a set of the light irradiation position and the light detection position which are disposed at a distance d1 and a logarithmic value of received light intensity measured by a set of the light irradiation position and the light detection position which are disposed at a distance d2, the sets of the light irradiation position and the light detection position being disposed on the living body.

Further, in order to achieve the above-mentioned object, the present invention provides an analysis method by an analysis unit that analyzes a detection signal obtained by detecting, at a light detection position on a living body, light which is irradiated from a light irradiation position on the living body and is propagated through the living body, the method including: obtaining, based on the detection signal, a value corresponding to a concentration change of an absorber inside the living body by using a gradient value with respect to a distance between the light irradiation position and the light detection position, the gradient value being a differential value in an amount of change between a logarithmic value of received light intensity measured by a set of the light irradiation position and the light detection position which are disposed at a distance d1 and a logarithmic value of received light intensity measured by a set of the light irradiation position and the light detection position which are disposed at a distance d2, the sets of the light irradiation position and the light detection position being disposed on the living body.

### Advantageous Effects of Invention

According to the present invention, it is possible to obtain a measurement signal proportional to a hemodynamic change at a deep part regardless of the SD distance.

### BRIEF DESCRIPTION OF DRAWINGS

FIG. 1 is a diagram illustrating an example of the relationship between partial optical path lengths and SD distances according to each example.

FIG. 2 is a diagram illustrating a disposition example of sets of light irradiation positions and light detection positions according to an example 1.

FIG. 3 is a diagram illustrating a disposition example of sets of light irradiation positions and light detection positions according to the example 1.

FIG. 4 is a diagram illustrating a disposition example of sets of light irradiation positions and light detection positions according to the example 1.

FIG. 5 is a diagram illustrating a configuration example of a device for the optical measurement of a living body according to the example 1.

FIG. 6 is an auxiliary explanatory diagram for obtaining ΔCdeep[t]·L0 according to an example 2.

FIG. 7 is an auxiliary explanatory diagram for obtaining ΔCdeep[t]·L0 according to the example 3.

FIG. 8 is a diagram illustrating the relationship between light irradiation positions, light detection positions, and measurement points according to an example 4.

FIG. 9 is a diagram illustrating a disposition example of light irradiation positions and light detection positions for measurement of a human according to an example 5.

### DESCRIPTION OF EMBODIMENTS

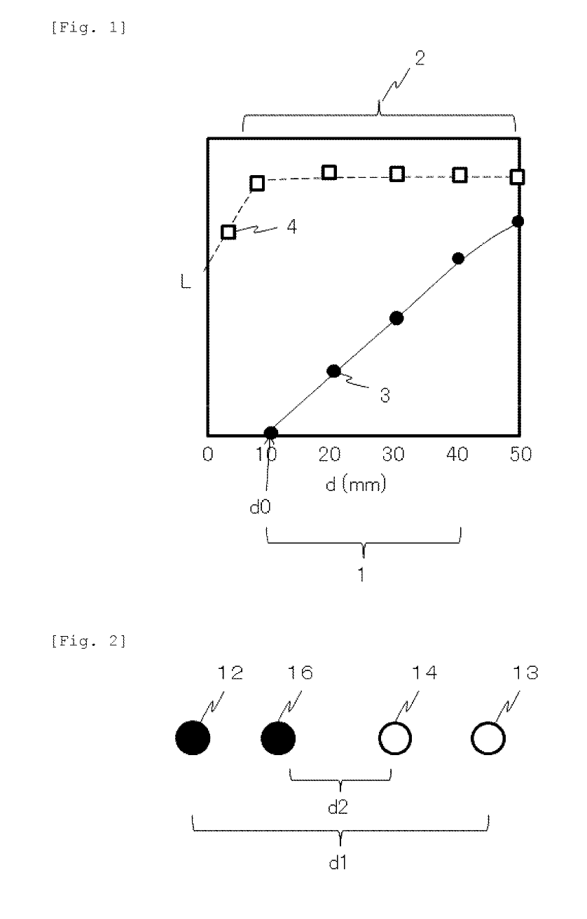

Hereinafter, the principle of the present invention will be described before sequentially describing various embodiments with reference to the drawings. FIG. 1 is a diagram illustrating an example of simulating a human head and calculating SD distance dependence in a partial optical path length 3 (Ldeep) of gray matter (deep part) and in a partial optical path length 4 (Lscalp) of scalp. In FIG. 1, the horizontal axis represents the SD distance d (mm), and the vertical axis represents the partial optical path length.

As can be seen from FIG. 1, the partial optical path length 3 (Ldeep) of the deep part has an X intercept d0 and a gradient L0, and can be approximated as a linear increase in a range 1 of the SD distance. In addition, the partial optical path length 4 (Lscalp) of the scalp can be approximated as no change in a range 2 of the SD distance. Therefore, in the range of the SD distance in which the range 1 of the SD distance and the range 2 of the SD distance overlap, assuming that the change with time in received light intensity measured at the SD distance d is I[d, t], a change in absorbance when employing a time point 0 as a starting point, that is, an amount of change ΔA[d, t] in a logarithmic value of received light intensity of a detector can be written as shown in Equation 1 by the modified Lambert-Beer equation.

In addition, here, in order to simplify explanation of the principle of the present invention, the following Equation 1 is described for the case of measuring total hemoglobin using an isosbestic point wavelength as an example. The case of spectroscopic measurement of oxygenated hemoglobin and deoxygenated hemoglobin using light having two or more wavelengths will be described in an example 2.

$$\begin{matrix}[ Equation 1 ] & \\ Δ A [ d , t ] = Log ( I [ d , 0 ] ) - Log ( I ( d , t ] ) = ɛΔ {C}_{deep} [ t ] \cdot {L}_{0} \cdot ( d - {d}_{0} ) + ɛΔ {C}_{scalp} [ t ] \cdot {L}_{scalp} & ( 1 )\end{matrix}$$

Here, ε represents the molecular extinction coefficient of total hemoglobin at the wavelength, and ΔCdeep and ΔCscalp respectively represent the total hemoglobin concentration change in the deep part and the scalp.

Subsequently, a differential value between ΔA[d1, t] measured at an SD distance d1 and ΔA[d2, t] measured at an SD distance d2 is taken, and the differential value is divided by the difference between the SD distances. Thus, Equation 2 is obtained.

$$\begin{matrix}[ Equation 2 ] & \\ Δ {C}_{deep} [ t ] \cdot {L}_{0} = \frac{( Δ A [ {d}_{1} , t ] - Δ A [ {d}_{2} , t ] )}{ɛ ( {d}_{1} - {d}_{2} )} & ( 2 )\end{matrix}$$

The right-hand side of Equation 2 represents that, when a logarithmic value of an amount of detected light at a certain time point is set as a starting point, at two SD distances, the amount of change in a logarithmic value of the amount of detected light at each time point is measured, and that a gradient value with respect to a differential SD distance which is a difference between an amount of change obtained by the measurement at a longer SD distance and an amount of measurement obtained by the measurement at a shorter SD distance, in other words, ΔAdiff/Δd which is a value proportional to a concentration change of an absorber inside a living body, is divided by ε. The new measurement amount obtained in this manner is proportional to the product of the hemoglobin concentration change in the deep part ΔCdeep and L0, and in the new measurement amount, the effect of blood flow (ΔCscalp·Lscalp) in skin is removed. Since L0 is the gradient of Ldeep with respect to d and can be regarded as a constant value, L0 is a value independent of the SD distance d. Here, L0 is dependent on the anatomical structure of the head and the optical structure depending on the distribution of optical properties. The value proportional to a concentration change of an absorber inside a living body can be referred to as a value corresponding to a concentration change of an absorber inside a living body.

The present invention is characterized by using the product of ΔCdeep and L0 as an indirect measurement amount. Although ΔCdeep has a dimension of concentration, L0 is a gradient and is a dimensionless amount. Thus, the measurement amount has a dimension of concentration. Further, in Equation 1, ΔCdeep·L0 can be replaced by the change Δ(Cdeep·L0) of the product of Cdeep and L0. In other words, even in a case where the optical structure of the head is changed, the amount including the change is considered as the indirect measurement amount.

In the case where Ldeep is for gray matter, from FIG. 1, it can be seen that the range of the SD distance where the range 1 of the SD distance and the range 2 of the SD distance overlap may be set to a range from approximately 10 mm to approximately 40 mm. Here, even when the upper limit of the range is changed from 40 mm to 50 mm, since the linearity is not greatly impaired, the upper limit of the range can be set to 50 mm or less, or 50 mm or more, depending on the allowable measurement accuracy. However, when the SD distance increases, the spatial resolution and the signal-to-noise ratio of a direct measurement amount decrease. Therefore, in an actual measurement, the SD distance may be selected according to the purpose. Preferably, the SD distance is set in a range from approximately 10 mm to approximately 50 mm. In Equation 1, although the logarithmic value of the amount of detected light at a certain time point 0 is used as a reference, the average value of the logarithmic values of the amount of detected light at a plurality of time points may be used as a reference.

Based on the above-described principle of the present invention, preferred embodiments of a device for the optical measurement of a living body according to the present invention have the following configuration. That is, a device for the optical measurement of a living body according to the present invention includes one or more light irradiators for irradiating a subject with light, one or more light detectors for detecting, at a light detection position on the subject, light which is irradiated to a light irradiation position on the subject from the one or more light irradiators and is propagated through the subject, and an analysis unit for analyzing a signal obtained by the one or more light detectors. Each of the light irradiators and the light detectors is disposed on the subject. There are at least two kinds of the SD distance which is defined as a distance between the light irradiation position and the light detection position, a long SD distance and a short SD distance. The SD distance has a value in a range in which the partial optical path length of the deep part can be approximated as a linear increase with respect to the SD distance. The analysis unit calculates a gradient value (ΔAdiff/Δd) with respect to the SD distance d, by taking a differential between a logarithmic value of a signal which is detected using the light irradiator and the light detector having a long SD distance and a logarithmic value of a signal which is detected using the light irradiator and the light detector having a short SD distance, and dividing the differential by a difference between the long SD distance and the short SD distance. The analysis unit obtains an indirect measurement signal (ΔCdeep·L0[t]) proportional to a hemodynamic change at the deep part, using the obtained gradient value. The indirect measurement signal is displayed on a display unit as a waveform with time or an image, and further stored in a storage unit.

Hereinafter, various embodiments of the present invention described above will be described step by step with reference to the drawings.

### EXAMPLE 1

The example 1 is an example of a device for the measurement of a living body, an analysis device, and an analysis method. The device for the optical measurement of a living body includes one or more light irradiators for irradiating a light irradiation position on the living body with light, one or more light detectors for detecting, at a light detection position on the living body, light which is propagated through the living body, and an analysis unit for analyzing a detection signal obtained by the one or more light detectors. The analysis unit obtains a value proportional to a concentration change of an absorber inside the living body by using a gradient value with respect to an SD distance based on the detection signal, as a value corresponding to the concentration change of the absorber inside the living body. The SD distance is a differential value in the amount of change between a logarithmic value of received light intensity measured by a set of the light irradiation position and the light detection position which are disposed at a distance d1 and a logarithmic value of received light intensity measured by a set of the light irradiation position and the light detection position which are disposed at a distance d2, the sets of the light irradiation position and the light detection position being disposed on a surface of tissue of the living body.

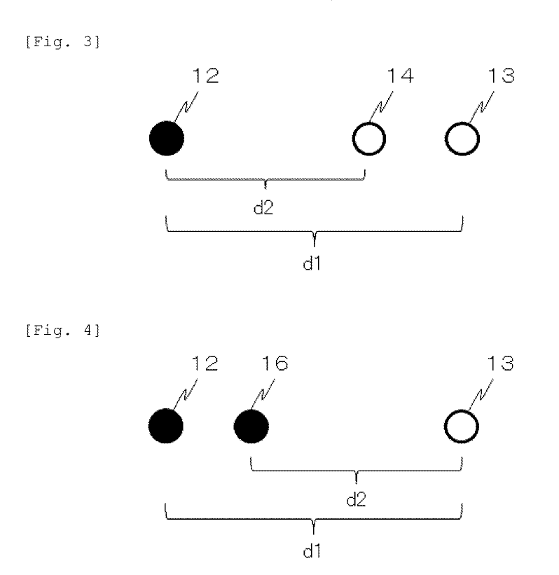

First, FIGS. 2 to 4 illustrate a disposition example of the sets of the light irradiation position and the light detection position which are used for calculating a value ΔAdiff/Δd corresponding to a concentration change of an absorber inside a living body, using the light irradiators and the light detectors in the device for the optical measurement of a living body according to the example 1. In FIG. 2, a first light irradiation position 12 and a first light detection position 13 are disposed at an SD distance d1 to form a pair, and a second light irradiation position 16 and a second light detection position 14 are disposed at an SD distance d2 to form a pair.

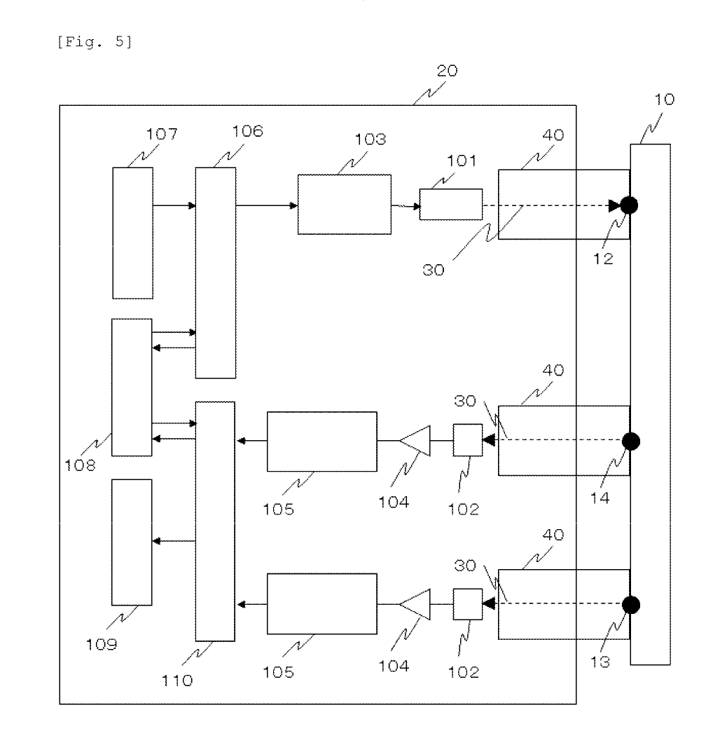

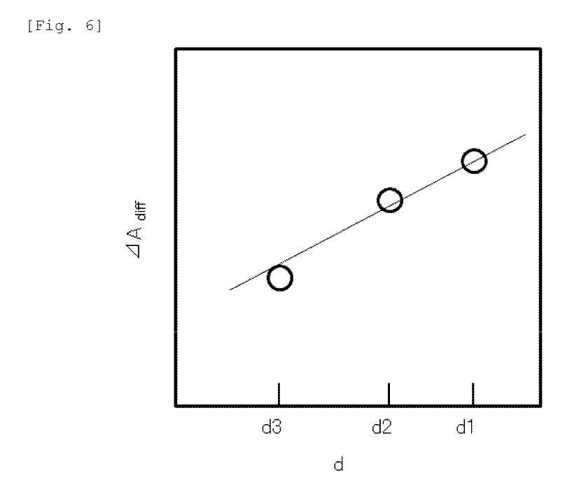

In FIG. 3, the light irradiation position 12 forms a pair with both of the first light detection position 13 and the second light detection position 14. That is, light irradiated from the light irradiation position 12 is detected at both of the first light detection position 13 and the second light detection position 14. On the contrary, in FIG. 4, light respectively irradiated from the first light irradiation position 12 and the second light irradiation position 16 is detected at one light irradiation position 13.

As illustrated in FIGS. 2 to 4, although the light irradiation position and the light detection position are preferably disposed in a straight line, in a region where a hemodynamic change can be regarded as approximately constant, light detection positions with different SD distances that are disposed in different directions can also be used.

FIG. 5 illustrates an example of the entire configuration of a device for the optical measurement of a living body according to the example 1. In a device for the optical measurement of a living body that makes light enter into a living body and detects light which is scattered, absorbed, and propagated in the living body and is output from the living body, light 30 irradiated from a light source 101 is entered into a living body, that is, a subject 10, via a waveguide 40, the light source 101 serving as one or more light irradiators included in a device main body 20. The light 30 is entered into the subject 10 from a light irradiation position 12, is passed and propagated through the subject 10, and then is detected by respective light detectors 102 from light detection positions 13 and 14 positioned away from the light irradiation position 12 via waveguides 40. The distance between the light irradiation position 12 and the light detection position 13 is d1, and the distance between the light irradiation position 12 and the light detection position 14 is d2. In FIG. 5, although a case where two light detection positions are provided is illustrated, three or more light detection positions may be provided.

Here, the one or more light sources 101 maybe a semiconductor laser (LD), a light emitting diode (LED), or the like, and the one or more light detectors 102 may be an avalanche photodiode (APD), a photodiode (PD), a photomultiplier tube (PMT), or the like. Further, the waveguides 40 may be an optical fiber, a glass, a light guide, or the like. The light source 101 is driven by a light source driving device 103. The output signals from the one or more light detectors are amplified by amplifiers 104, and are converted from analog signals to digital signals by analog-to-digital converters 105. The values of the converted signals are processed by an analysis unit 110, and the processed results are displayed on a display unit 109 and are stored in a storage unit 108. A control unit 106 controls the light source driving device 103 based on input of a condition or the like from an input unit 107 and data of the storage unit 108.

Needless to say, the control unit 106, the input unit 107, the storage unit 108, the display unit 109, and the analysis unit 110 of the device for the optical measurement of a living body that are illustrated in FIG. 5 can be realized by a general computer configuration such as a personal computer (PC), for example. In particular, the control unit 106 and the analysis unit 110 can be realized by a program execution in a central processing unit (CPU) of a PC. The storage unit 108 can store various data measured or calculated, and various programs for realizing functions of the control unit 106 and the analysis unit 110.

The analysis unit 110 that can be realized by a CPU or the like executes an analysis based on the signals detected by the light detectors 102. Specifically, the analysis unit 110 receives the digital signals obtained by the conversion in the analog-to-digital converters 105, and obtains, based on the digital signals, respectively, (ΔCoxyL0)deep and (ΔCdeoxyL0)deep for oxygenated hemoglobin in a deep part and deoxygenated hemoglobin in a deep part, by the following calculation. In a case where two wavelengths λ1 and λ2 are used as the output of the light source 101, Equation 2 can be expressed as follows.

$$\begin{matrix}[ Equation 3 ] & \\ ( \begin{matrix}Δ {A}^{λ 1} [ {d}_{1} , t ] - Δ {A}^{λ 1} [ {d}_{2} , t ] \\ Δ {A}^{λ 2} [ {d}_{1} , t ] - Δ {A}^{λ 2} [ {d}_{2} , t ]\end{matrix} ) = ( \begin{matrix}{ɛ}_{oxy}^{λ 1} & {ɛ}_{deoxy}^{λ 1} \\ {ɛ}_{oxy}^{λ 2} & {ɛ}_{deoxy}^{λ 2}\end{matrix} ) \times ( \begin{matrix}{( Δ {C}_{oxy} L )}_{deep} [ {d}_{1} , t ] + {( Δ {C}_{oxy} L )}_{scalp} [ {d}_{1} , t ] - {( Δ {C}_{oxy} L )}_{deep} [ {d}_{2} , t ] - {( Δ {C}_{oxy} L )}_{scalp} [ {d}_{2} , t ] \\ {( Δ {C}_{deoxy} L )}_{deep} [ {d}_{1} , t ] + {( Δ {C}_{deoxy} L )}_{scalp} [ {d}_{1} , t ] - {( Δ {C}_{deoxy} L )}_{deep} [ {d}_{2} , t ] - {( Δ {C}_{deoxy} L )}_{scalp} [ {d}_{2} , t ]\end{matrix} ) = ( \begin{matrix}{ɛ}_{oxy}^{λ 1} & {ɛ}_{deoxy}^{λ 1} \\ {ɛ}_{oxy}^{λ 2} & {ɛ}_{deoxy}^{λ 2}\end{matrix} ) ( \begin{matrix}{( Δ {C}_{oxy} [ t ] {L}_{0} ( {d}_{1} - {d}_{2} ) )}_{deep} \\ {( Δ {C}_{deoxy} [ t ] {L}_{0} ( {d}_{1} - {d}_{2} ) )}_{deep}\end{matrix} ) & ( 3 )\end{matrix}$$

Here, the subscripts oxy and deoxy in the respective parameters represent that the parameters correspond to oxygenated hemoglobin and deoxygenated hemoglobin. ε with the subscript λ1 and ε with the subscript λ2 represent the molecular extinction coefficients of hemoglobin at the respective wavelengths. When equation 3 solved for ΔCdeepL0, equation 4 is obtained.

$$\begin{matrix}[ Equation 4 ] & \\ ( \begin{matrix}{( Δ {C}_{oxy} [ t ] {L}_{0} )}_{deep} \\ {( Δ {C}_{deoxy} [ t ] {L}_{0} )}_{deep}\end{matrix} ) = {( \begin{matrix}{ɛ}_{oxy}^{λ 1} & {ɛ}_{deoxy}^{λ 1} \\ {ɛ}_{oxy}^{λ 2} & {ɛ}_{deoxy}^{λ 2}\end{matrix} )}^{- 1} ( \begin{matrix}Δ {A}^{λ 1} [ {d}_{1} , t ] - Δ {A}^{λ 1} [ {d}_{2} , t ] / ( {d}_{1} - {d}_{2} ) \\ Δ {A}^{λ 2} [ {d}_{1} , t ] - Δ {A}^{λ 2} [ {d}_{2} , t ] / ( {d}_{1} - {d}_{2} )\end{matrix} ) & ( 4 )\end{matrix}$$

In the right side of equation 4, the matrix on the right side is a matrix including ΔAdiff/Δd as an element to be used in the case of two wavelength measurement. Equation 4 corresponds to equation in the case of using the above-described isosbestic point wavelength.

Here, although the control unit 106 is described to perform all of driving of the light source 101, gain control of the light detectors 102, and processing of signals from the analog-to-digital converters 105, the same function can be realized by providing separate control units and providing means for integrating the separate control units. In addition, in addition, here, although the calculation is performed after the digital conversion, the calculation may be performed in an analog manner using a logarithmic amplifier or a differential amplifier. Further, here, although light is propagated using the optical waveguides 40 between the light source 101 and the subject 10 and between the light detector 102 and the subject 10, the light source and the light detector may be directly brought into contact with the living body.

In the present example, although the case of using a light source with two wavelengths is described, the same calculation can be performed for the case of using a light source with one wavelength and the case of using a light source with three or more wavelengths. In addition, although the measurement for one set of the light detection positions is described in the present example, similar to a device in the related art, the measurement for a plurality of sets of the light detection positions may be performed and imaged. Further, in the present example, although a case where a plurality of light detectors are provided for one light irradiator is described, a plurality of light irradiators may be used for one light detector. Furthermore, a plurality of sets of the light irradiator and the light detector that have different SD distances may be used without sharing the light irradiator and the light detector between the sets. Here, it is possible to reduce the number of components by sharing the light irradiator and the light detector between the sets.

In a case where an amount of output light of the light irradiator changes with time, the amount of output light of the light irradiator is set to I0[t], and when describing equation 1 using an indirect measurement amount ΔCL in the related art, the following equation is obtained.

$$\begin{matrix}[ Equation 5 ] & \\ Δ A [ d , t ] = Log ( I [ d , 0 ] ) - Log ( I ( d , t ] ) = Log ( {I}_{0} [ 0 ] ) - Log ( {I}_{0} [ t ] ) + ɛΔ C [ t ] \cdot L & ( 5 )\end{matrix}$$

In a case where the amount of output light of the light irradiator changes with time, in the right side of equation 5, since the first term and the second term are present, the first term and the second term are measured as a change in ΔCL. Therefore, in a device in the related art, control means for stabilizing the amount of output light of the light source, that is, a circuit or the like for detecting a part of the amount of output light of the light source and applying negative feedback control is necessary. In the present example, particularly, in a case where a configuration in which a plurality of light detectors are used for one light irradiator is employed, for example, even when there is a change in an amount of irradiation light of the light source as in equation 5, the terms (I0[0] and I0[t]) of the amount of irradiation light of the light source are canceled by taking the difference between ΔA[d1, t] and ΔA[d2, t]. Thus, equation 2 is obtained. This means that an indirect measurement amount ΔCdeep·L0, which is a feature of the present example, is not influenced by a change in irradiation light intensity. Therefore, by employing a configuration in which a plurality of light detectors are used for one light irradiator, noise and fluctuation in output of the light source can be canceled, and thus it is possible to improve the measurement accuracy. As a result, there is no need for control means for stabilizing the amount of output light of the light source, and thus there is an advantage in that it is possible to reduce the size and the cost of the device.

According to the present example, it is possible to obtain a measurement signal proportional to a hemodynamic change at a deep part regardless of SD distances. Therefore, there is no need to dispose, at an exact distance, an optical fiber and an optical element which are normally used as light irradiation means and light detection means, and thus the degree of freedom of disposition increases. Accordingly, it is possible to provide disposition according to the position of the brain to be measured, regardless of the head size or the head shape of the subject. Furthermore, there is an advantage in that it is possible to compare the measurement results between devices with different SD distances or between different measurement conditions.

### EXAMPLE 2

Next, an example of a device for the optical measurement of a living body using detection signals measured at three or more different SD distances, will be described as an example 2. In the example 1, the detection signals measured at two different SD distances d1 and d2 are used. The present example shows that the disposition can be similarly made even in case of three or more different SD distances.

A calculation in the case of using three SD distances d1, d2, and d3 as a set will be described. Three light detectors are disposed at SD distances d1, d2, and d3 from one light irradiator. When combining two light detectors among the three light detectors, there are three combinations d1−d2, d2−d3, and d1−d3. Thus, for each subset, three ΔAdiff[t]/Δd are obtained. The average value of these three values is set as a measurement value ΔCdeep[t]·L0 of the set. Accordingly, it is possible to reduce a measurement error. In the present example, although a description of three SD distances is given, the calculation can be similarly performed even in the case of using four or more SD distances as a set. Here, Δd of each subset may be the same value or different values. Further, in a case where there are a plurality of sets, Δd of each set may be the same value or different values.

### EXAMPLE 3

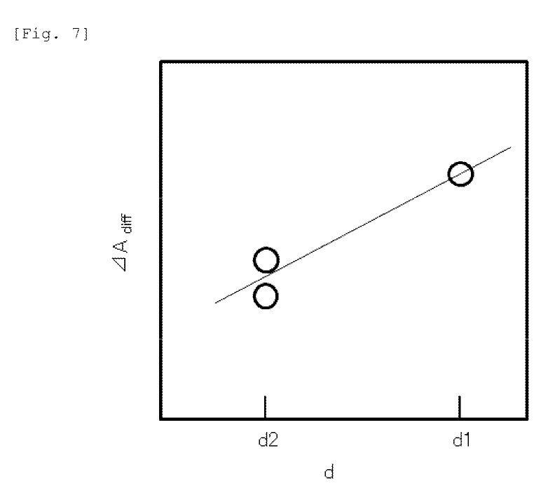

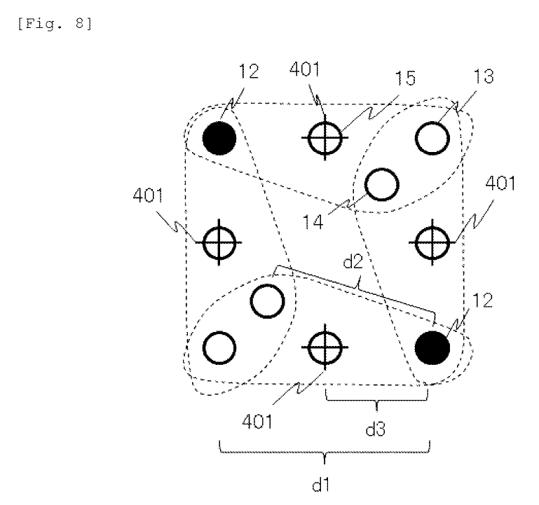

In the present example, another calculation in the case of using three SD distances d1, d2, and d3 as a set will be described. As illustrated in FIG. 6, at each time point t, measured values at three SD distances are plotted on a graph in which d is set as a horizontal axis and ΔAdiff is set as a vertical axis, and a gradient value (ΔAdiff/Δd) of the measured values is obtained by linear regression. Then, a value obtained by dividing the gradient value by ε is set as a measurement value ΔCdeep[t]·L0 of the set. Even in this case, there is an advantage in that it is possible to reduce a measurement error similarly to the example 3. In FIG. 6, although a case where d1, d2, and d3 are different from each other is described, some of d1, d2, and d3 may have the same value (d2=d3) as illustrated in FIG. 7. Further, the calculation can be similarly performed even in the case of using four or more SD distances as a set.

### EXAMPLE 4

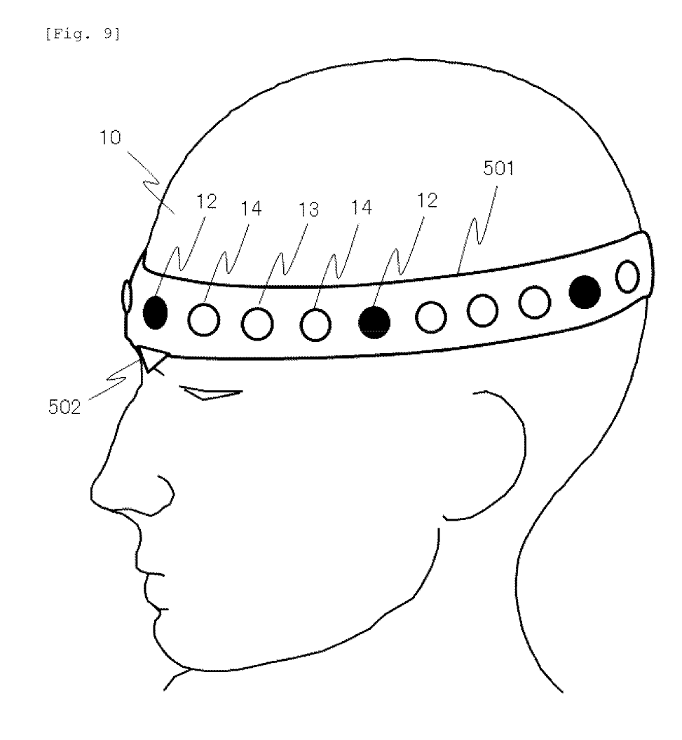

As an example 4, an example of a method for preferably imaging the measurement value measured by the analysis unit or the like in the device for the optical measurement of a living body will be described. FIG. 8 is a diagram explaining a method for imaging the measurement value ΔCdeep·L0. In FIG. 8, light detectors 13, 14, and 15 are disposed at SD distances d1, d2, and d3 from light irradiator 12, and four sets surrounded by broken lines are formed. FIG. 8 illustrates an example of reducing the number of the light detectors in a manner by which the light detector 14 is not disposed on the same straight line on which other light detectors are disposed, and by which the light detectors are shared between the sets such that the distance between the light detector and one light irradiator is the same as the distance between the light detector and the other light irradiator. In a region between the light irradiation position and the light detection position in the set, it can be said that hemodynamics in skin and hemodynamics in brain are respectively uniform, and thus ΔAdiff can be regarded as a value reflecting information between the light irradiation position and the light detection position furthest from the light irradiation position.

Therefore, an approximate midpoint between the light detection position and the light irradiation position of a set of the light detection position and the light irradiation position that is the longest in SD distance can be represented as a measurement point 401 of the set. In this manner, when the measurement value ΔCdeep·L0 at the measurement point (+ intersection point) is obtained for each set, the measurement value is processed by a program in the analysis unit 110 illustrated in FIG. 5 that is realized by a CPU and the like. The measurement value between the measurement points is interpolated as necessary. Thus, the measurement value is displayed an image similarly to the case in the related art. Here, although the measurement point 401 and the position of the light detector 14 overlap each other, this is because the case where the light detector 15 is disposed at the midpoint between the light irradiator 12 and the light detector 13 is illustrated. The light detector 15 is not necessarily disposed at the midpoint, and in that case, the measurement point 401 does not match with the position of the light detector 15.

### EXAMPLE 5

As an example 5, an example for a preferred disposition of the light irradiator and the light detector in a device for the optical measurement of a living body will be described. Since the configuration of the main body of the device for the optical measurement of a living body is the same as that of the device according to other examples, the description thereof is omitted here.

As illustrated in FIG. 9, in the present example, light irradiators 12 and light detectors 13 and 14 are combined by a holding unit 501 made of an expandable mechanism or an expandable member. The holding unit 501 is expanded according to the shape of the head of a subject, and thus SD distances are expanded and set. A marker 502 attached to the holding unit 501 is positioned at the nose root of a subject, and another marker (not illustrated) attached to the holding unit 501 is positioned to the occipital protuberance. Thus, light irradiation positions and light detection positions can be disposed at positions where the head circumference is divided along the shape of the head of the subject. As an external pointer, in addition to the nose root and the occipital protuberance, an earlobe, a median central portion, or the like is generally used.

In a device in the related art, in order to keep SD distances constant, light irradiators and light detectors are combined by a non-expandable member such that the distances between light irradiation positions and light detector positions do not change. For this reason, for subjects with different head sizes and different head shapes, since the measurement positions are displaced, the brain area to be measured is displaced. Thus, in some cases, additional work that separately measures the relative positional relationship between light irradiation positions, light detection positions, and external pointers of the subject's head, and that estimates the brain area to be measured, is performed.

On the other hand, in the device according to the present example, since it is not necessary to keep the SD distances constant, it is possible to dispose light irradiation positions and light detector positions at positions relative to the head shape of the subject. The position of the brain area can be estimated by the positions relative to the external pointer of the subject's head. Thus, according to the configuration of the present example, there is an advantage in that it is possible to easily estimate the measurement position corresponds to which portion of the brain. In addition, the measurement position is standardized based on the external pointer, and thus there is also an advantage in that it is possible to compare and calculate measurement data obtained at the same relative position regardless of the head shape of the subject.

Further, in simultaneous measurement of a brain area and brain waves, measurement positions of brain waves are disposed at positions relative to the external pointer as a reference, whereas optical probes of NIRS need to be disposed at absolute positions with fixed SD distances. Thus, there is a problem that it is difficult to dispose brain wave electrodes and optical probes since the disposition positions of the brain wave electrodes interfere with the disposition positions of the optical probes, and that the positional relationship between the measurement positions of brain waves and the measurement positions of NIRS is displaced for each subject. The light irradiation positions and the light detector positions are disposed in the same manner as the international 10-20 method commonly used for brain wave electrode disposition or in a manner based on the international 10-20 method. Thus, there is an advantage in that it is possible to prevent the interference and easily perform the simultaneous measurement of brain waves and NIRS. In the present example, although the disposition of the light irradiation positions and the light detector positions which make one round of a head is described, even in measurement of the entire head or partial measurement of a head, the light irradiation positions and the light detector positions can be disposed in the same manner.

The present invention is not limited to the above-described examples, and includes various modified examples. For example, the above-described examples have been described in detail for abetter understanding of the present invention, and are not necessarily limited to those including all the configurations described above.

In addition, a part of the configuration of an example can be replaced by the configuration of another example, and the configuration of an example can be added to the configuration of another example. Further, addition of another configuration, omission, substitution in apart of the configuration of each example can be made.

Furthermore, needless to say, some or all of the above-described configurations, functions, processing units, and the like may be realized by hardware using, for example, design of integrated circuits or the like, and may be realized by software using program creation. In the present specification, first, the difference in absorbance measured at different SD distances is calculated, and then ΔCdeep·L0 is obtained using the modified Lambert-Beer equation. However, first, ΔC·L may be obtained based on the absorbance measured at each SD distance using the modified Lambert-Beer equation, and then ΔCdeep·L0 may be obtained by calculating the difference in absorbance. For example, in a case where SD distances are d1 and d2, ΔCdeep·L0 is expressed as equation 6.

$$\begin{matrix}[ Equation 6 ] & \\ \frac{Δ C \cdot L [ {d}_{1} , t ] - Δ C \cdot L [ {d}_{2} , t ]}{{d}_{1} - {d}_{2}} = \frac{Δ {C}_{deep} [ t ] \cdot {L}_{0} \cdot ( {d}_{1} - {d}_{0} ) + Δ {C}_{scalp} [ t ] \cdot {L}_{scalp} - Δ {C}_{deep} [ t ] \cdot {L}_{0} \cdot ( {d}_{2} - {d}_{0} ) - Δ {C}_{scalp} [ t ] \cdot {L}_{scalp}}{{d}_{1} - {d}_{2}} = Δ {C}_{deep} [ t ] \cdot {L}_{0} & ( 6 )\end{matrix}$$

### REFERENCE SIGNS LIST

1: range in which partial optical path length of gray matter is linear

2: range in which partial optical path length of scalp is constant

3: partial optical path length of gray matter

4: partial optical path length of scalp

10: subject

12, 16: light irradiation position

13, 14, 15: light detection position

20: device main body

30: light

40: optical waveguide

50: light irradiator

60, 102: light detector

101: light source

103: light source driving device

104: amplifier

105: analog-to-digital converter

106: control unit

107: input unit

108: storage unit

109: display unit

110: analysis unit

401: measurement point

501: holding unit

502: marker

1. A device for the optical measurement of a living body, comprising:
one or more light irradiators that irradiate a light irradiation position on the living body with light;
one or more light detectors that detect, at a light detection position on the living body, light which is propagated through the living body; and
an analysis unit that analyzes a detection signal obtained by the one or more light detectors,
wherein the analysis unit
obtains, based on the detection signal, a value corresponding to a concentration change of an absorber inside the living body by using a gradient value with respect to a distance between the light irradiation position and the light detection position, the gradient value being a differential value in an amount of change between a logarithmic value of received light intensity measured by a set of the light irradiation position and the light detection position which are disposed at a distance d1 and a logarithmic value of received light intensity measured by a set of the light irradiation position and the light detection position which are disposed at a distance d2 or being a differential value in an amount of change of hemoglobin, the sets of the light irradiation position and the light detection position being disposed on a surface of tissue of the living body.

2. The device for the optical measurement of a living body according to claim 1,
wherein the distance d1 and the distance d2 are in a range in which partial optical path lengths at a shallow part of the living body can be regarded as the same and partial optical path lengths at a deep part of the living body are proportional to the distance between the light irradiation position and the light detection position.

3. The device for the optical measurement of a living body according to claim 1,
wherein the distance d1 and the distance d2 are set in a range from approximately 10 mm to approximately 50 mm.

4. The device for the optical measurement of a living body according to claim 1,
wherein the analysis unit
sets, among at least two sets of the light irradiation position and the light detection position that are used for obtaining the value corresponding to the concentration change of the absorber inside the living body, an approximate midpoint between the light irradiation position and the light detection position of a set of the light irradiation position and the light detection position that is the longest in distance, as a measurement point of the value corresponding to the concentration change of the absorber, and images the value corresponding to the concentration change of the absorber or a waveform with time of the value.

5. The device for the optical measurement of a living body according to claim 1,
wherein the set of the light irradiation position and the light detection position is configured with the one light irradiator and the light detectors which are respectively disposed at the distance d1 and at the distance d2 from the one light irradiator.

6. The device for the optical measurement of a living body according to claim 1,
wherein the light irradiator and the light detector are combined by an expandable holding unit.

7. An analysis device comprising:
an analysis unit that analyzes a detection signal obtained by detecting, at a light detection position on a living body, light which is irradiated from a light irradiation position on the living body and is propagated through the living body,
wherein the analysis unit
obtains, based on the detection signal, a value corresponding to a concentration change of an absorber inside the living body by using a gradient value with respect to a distance between the light irradiation position and the light detection position, the gradient value being a differential value in an amount of change between a logarithmic value of received light intensity measured by a set of the light irradiation position and the light detection position which are disposed at a distance d1 and a logarithmic value of received light intensity measured by a set of the light irradiation position and the light detection position which are disposed at a distance d2, the sets of the light irradiation position and the light detection position being disposed on the living body.

8. The analysis device according to claim 7,
wherein the distance d1 and the distance d2 are in a range in which partial optical path lengths at a shallow part of the living body can be regarded as the same and partial optical path lengths at a deep part of the living body are proportional to the distance between the light irradiation position and the light detection position.

9. The analysis device according to claim 7,
wherein the distance d1 and the distance d2 are set in a range from approximately 10 mm to approximately 50 mm.

10. The analysis device according to claim 7,
wherein the analysis unit
sets, among at least two sets of the light irradiation position and the light detection position that are used for obtaining the value corresponding to the concentration change of the absorber inside the living body, an approximate midpoint between the light irradiation position and the light detection position of a set of the light irradiation position and the light detection position that is the longest in distance, as a measurement point of the value corresponding to the concentration change of the absorber, and images the value corresponding to the concentration change of the absorber or a waveform with time of the value.

11. The analysis device according to claim 7, further comprising:
a display unit,
wherein the display unit displays the value that is imaged and corresponds to the concentration change of the absorber, or a waveform with time of the value.

12. An analysis method by an analysis unit that analyzes a detection signal obtained by detecting, at a light detection position on a living body, light which is irradiated from a light irradiation position on the living body and is propagated through the living body, the method comprising:
obtaining, based on the detection signal, a value corresponding to a concentration change of an absorber inside the living body by using a gradient value with respect to a distance between the light irradiation position and the light detection position, the gradient value being a differential value in an amount of change between a logarithmic value of received light intensity measured by a set of the light irradiation position and the light detection position which are disposed at a distance d1 and a logarithmic value of received light intensity measured by a set of the light irradiation position and the light detection position which are disposed at a distance d2, the sets of the light irradiation position and the light detection position being disposed on the living body.

13. The analysis method according to claim 12,
wherein the distance d1 and the distance d2 are in a range in which partial optical path lengths at a shallow part of the living body can be regarded as the same and partial optical path lengths at a deep part of the living body are proportional to the distance between the light irradiation position and the light detection position.

14. The analysis method according to claim 12,
wherein the distance d1 and the distance d2 are set in a range from approximately 10 mm to approximately 50 mm.

15. The analysis method according to claim 12,
wherein the analysis unit
sets, among at least two sets of the light irradiation position and the light detection position that are used for obtaining the value corresponding to the concentration change of the absorber inside the living body, an approximate midpoint between the light irradiation position and the light detection position of a set of the light irradiation position and the light detection position that is the longest in distance, as a measurement point of the value corresponding to the concentration change of the absorber, and images the value corresponding to the concentration change of the absorber or a waveform with time of the value.
